(19) United States
(12) Patent Application Publication
(10) Pub. No.: US 2020/0069285 A1
(43) Pub. Date: Mar. 5, 2020

(54) SYSTEM AND METHOD FOR ULTRASOUND NAVIGATION

(71) Applicant: GENERAL ELECTRIC COMPANY, SCHENECTADY, NY (US)

(72) Inventors: Pavan Kumar Annangi, Bangalore (IN); Chandan Kumar Aladahalli, Bangalore (IN); Krishna Seetharam Shriram, Bangalore (IN); Prasad Sudhakar, Bangalore (IN)

(21) Appl. No.: 16/118,466

(22) Filed: Aug. 31, 2018

(51) Int. Cl. A61B 8/00; A61B 34/10; A61B 34/20; A61B 8/08

(52) U.S. Cl. CPC A61B 8/4254; A61B 34/10; A61B 2034/2063; A61B 8/5207; A61B 2034/105; A61B 34/20

(57) ABSTRACT

A method for ultrasound imaging is presented. The method includes acquiring at least one image of a subject, determining a current position of an ultrasound probe on a body surface of the subject based on the image, identifying anatomical regions of interest in the image, quantifying the image to determine suitability of the image to one or more scan planes corresponding to a clinical protocol, generating a personalized anatomical model of the subject based on a current position of the ultrasound probe, the identified anatomical regions of interest, and the quantification of the image, computing a desired trajectory of the ultrasound probe from the current location to a target location based on the clinical protocol, communicating a desired movement of the ultrasound probe based on the computed trajectory, moving the ultrasound probe along the computed trajectory based on the communicated desired movement to acquire images of the subject.

### BACKGROUND

Embodiments of the present specification generally relate to ultrasound imaging and more specifically to a system and method for clinician independent guidance in an ultrasound imaging system.

Ultrasound imaging provides a relatively inexpensive method of imaging. During the process of ultrasound scanning, a clinician attempts to capture a view of a certain anatomy which confirms/negates a particular medical condition. Once the clinician is satisfied with the quality of a view or a scan plane, the image is frozen to proceed to a measurement phase. For example, ultrasound images are routinely used to assess gestational age (GA) and weight of a fetus or to monitor cardiac health of a patient. Ultrasound measurements of specific features of fetal anatomy such as the head, abdomen or the femur from two-dimensional (2D) or three-dimensional (3D) image data are used in the determination of GA, assessment of growth patterns and identification of anomalies. Similarly, for cardiac applications, thicknesses of cardiac walls are routinely measured by cardiologists to check for cardiomyopathy.

Recent developments in ultrasound imaging have led to current state of the art ultrasound devices that boast of relatively high image resolutions and ease of use. These developments have in turn led to increased use of ultrasound for clinical research as well as day to day point of care practice. Consequently, the use of ultrasound imaging has been steadily increasing over the years. Moreover, the improved ultrasound technology has led to higher frequency ultrasound probes that are well-suited for imaging relatively shallow anatomical structures, as is generally the case for musculoskeletal imaging.

Notwithstanding the various advantages of ultrasound, an important factor that restricts the use of ultrasound at the point of care has been the fact that performing ultrasound scanning requires experience and training of a clinician. In addition, use of ultrasound leads to subjective diagnosis even among relatively skilled ultrasound practitioners such as sonographers. More particularly, image acquisition is quite a challenging problem for sonographers. Currently, image acquisition takes anywhere between 1 to 5 minutes for each correct scan plane acquisition and more so for novice clinicians. The other challenge the less experienced clinicians/sonographers face is the ability to correctly identify acceptable scan plane frames. It is also desirable for the clinicians to have an understanding of how far they are from correct scan plane. Moreover, ultrasound images are subject to both patient and clinician variability. Also, determining a quality of an image frame is fraught with challenges. Particularly, pixel intensities in the images vary significantly with different gain settings.

Furthermore, clinician variability also limits reproducibility of ultrasound imagery and measurement, There are multiple reasons for the inter-clinician variability. For example, two-dimensional (2D) echocardiography visualizes only a cross-sectional slice of a three-dimensional structure, commonly referred to as the scan plane, Even small changes in positioning of the transducer, which has six degrees of freedom, may lead to significant changes in the scene visualized, which may in turn lead to incorrect measurements. In addition, sub-optimal ultrasound image settings such as gain, time-gain compensation may decrease the ability to visualize the internal structures of the human body.

Early efforts at improving robustness and accuracy of clinical workflow have tended to focus on semi-automated methods for segmenting an anatomical region of interest. However, these processes tend to be time-consuming. Additionally, use of these techniques may entail user intervention or call for a trained sonographer. These techniques may also be subject to clinician variability or may be prone to false detection. In remote or rural markets, it may be particularly difficult to obtain services of a trained ultrasonographer or ultrasound technician, causing remote regions to be poorly served or underserved.

### BRIEF DESCRIPTION

In accordance with aspects of the present specification, a method for ultrasound imaging is presented. The method includes acquiring, via an ultrasound probe, at least one image of a subject. Further, the method includes determining in real-time, via a navigation platform, a current position of an ultrasound probe on a body surface of a subject based on the at least one image. In addition, the method includes identifying in real-time, via the navigation platform, one or more anatomical regions of interest in the at least one image. The method also includes quantifying in real-time, via the navigation platform, the at least one image to determine suitability of the at least one image to one or more scan planes corresponding to a determined clinical protocol. Moreover, the method includes generating in real-time, via the navigation platform, a personalized anatomical model of the subject based on a current position of the ultrasound probe, the identified one or more anatomical regions of interest, and the quantification of the at least one image. Also, the method includes computing in real-time, via the navigation platform, a desired trajectory of the ultrasound probe from the current location to a target location based on the determined clinical protocol. Furthermore, the method includes communicating in real-time, via the navigation platform, a desired movement of the ultrasound probe based on the computed trajectory. Additionally, the method includes moving the ultrasound probe along the computed trajectory based on the communicated desired movement to acquire images of the subject, where the acquired images include a desired anatomical region of interest.

In accordance with another aspect of the present specification, a system is presented. The system includes a navigation platform, where the navigation platform includes an anatomy positioning unit configured to determine in real-time a current position of an ultrasound probe on a body surface of a subject based on at least one image, an anatomy cognition unit configured to identify in real-time one or more anatomical regions of interest in the at least one image, a scan plane scoring unit configured to quantify in real-time the at least one image to determine suitability of the at least one image to one or more scan planes corresponding to a determined clinical protocol, a subject modeling unit configured to generate in real-time a personalized anatomical model of the subject based on a current position of the ultrasound probe, previous positions of the ultrasound probe, the identified one or more anatomical regions of interest, and the quantification of the at least one image, or combinations thereof, a guidance unit configured to compute in real-time a desired trajectory of the ultrasound probe from the current location to a target location based on the determined clinical protocol, and a feedback unit configured to communicate in real-time a desired movement of the ultrasound probe based on the computed trajectory.

In accordance with yet another aspect of the present specification, an imaging system is presented. The imaging system includes an acquisition subsystem configured to acquire at least one image corresponding to a subject. Moreover, the imaging system includes a processing subsystem in operative association with the acquisition subsystem and configured to process the at least one image, where the processing subsystem comprises a navigation platform configured to determine in real-time a current position of an ultrasound probe on a body surface of the subject based on the at least one image, identify in real-time one or more anatomical regions of interest in the at least one image, quantify in real-time the at least one image to determine suitability of the at least one image to one or more scan planes corresponding to a determined clinical protocol, generate in real-time a personalized anatomical model of the subject based on a current position of the ultrasound probe, previous positions of the ultrasound probe, the identified one or more anatomical regions of interest, and the quantification of the at least one image, or combinations thereof, compute in real-time a desired trajectory of the ultrasound probe from the current location to a target location based on the determined clinical protocol, and communicate in real-time a desired movement of the ultrasound probe based on the computed trajectory.

### DRAWINGS

These and other features, aspects, and advantages of the present disclosure will become better understood when the following detailed description is read with reference to the accompanying drawings in which like characters represent like parts throughout the drawings, wherein:

FIG. 1 is a diagrammatical illustration of a system for ultrasound imaging, in accordance with aspects of the present specification;

FIG. 2 is a diagrammatical illustration of one embodiment of a navigation platform for use in the system of FIG. 1, in accordance with aspects of the present specification;

FIG. 3 depicts a flow chart illustrating an exemplary method for ultrasound imaging, in accordance with aspects of the present specification;

FIGS. 4(a)-4(c) depict diagrammatic illustrations of one example of the exemplary method for ultrasound imaging of FIG. 3, in accordance with aspects of the present specification; and

FIG. 5 is a diagrammatical illustration of an ultrasound imaging system for use in the system of FIG. 1.

### DETAILED DESCRIPTION

Ultrasound imaging is being increasingly used to image anatomical regions of interest in a patient. As will be appreciated, more than other medical imaging modalities, in ultrasound imaging, efficacy of acquisition of relevant images for clinical diagnosis is highly dependent on the skill of the clinician/operator, in particular, clinically relevant outcomes depend on the skill level of the clinician in ultrasound scanning. Low-skilled clinicians often struggle to identify the anatomy, which is to be followed by the tasks of greater complexity, namely the identification of the right scan plane and biometry. Various systems and methods for ultrasound imaging of the present application present a robust, intelligent technique that will enable clinicians with varying skill levels to be effective in ultrasonography, More particularly, the systems and methods described hereinafter provide actionable instructions in terms of probe movements to aid the clinician in consistently arriving at the clinical gold standard will lead to consistent outcomes.

It may be noted that although the various systems and methods are described in the context of a medical imaging system, these systems and methods may also be used in the imaging of non-living objects such as but not limited to pipes, tubes, luggage, packages, and the like. Furthermore, the systems and methods of imaging are described with reference to providing assistance to a clinician such as a clinician, sonographer, or a radiologist in locating the right kidney of a subject. However, the present systems and methods may also find application in assisting the clinician in locating other anatomical regions in the subject.

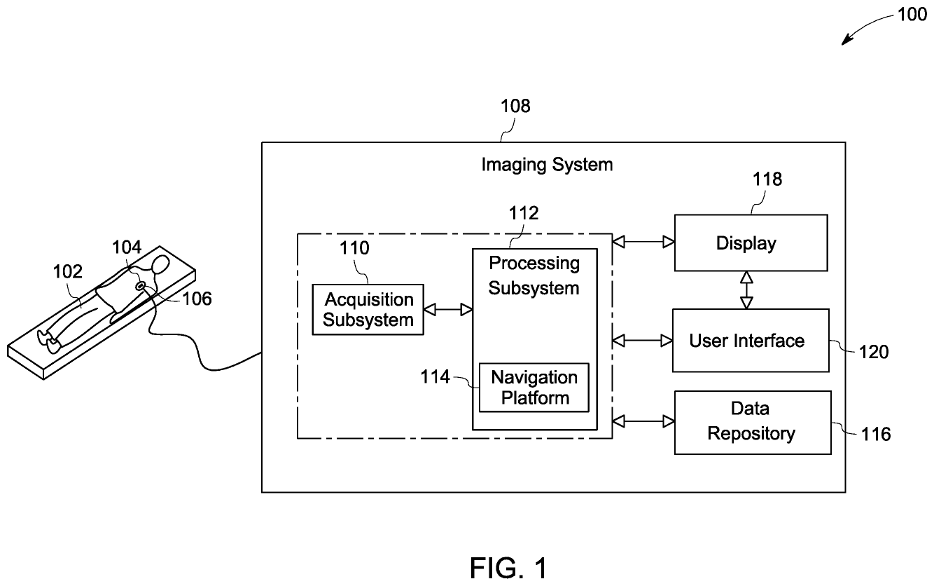

FIG. 1 is a block diagram of an exemplary system 100 for use in diagnostic imaging, in accordance with aspects of the present specification. More particularly, the system 100 is configured to aid a clinician in imaging a patient 102 to deliver consistent clinical outcomes irrespective of the skill level of the clinician such as a sonographer and/or a medical practitioner.

During imaging, the clinician typically positions an ultrasound probe on or about a region of interest in a patient 102 being imaged. In one example, the patient 102 may be positioned in a supine position on a patient support. Furthermore, an image acquisition device 104 that is operatively coupled to a medical imaging system 108 may be used to acquire image data corresponding to an object or region of interest in the patient 102. In one embodiment, the image acquisition device 104 may be an ultrasound probe. Additionally, in one example, the medical imaging system 108 is an ultrasound imaging system. The ultrasound imaging system 108 may be configured to receive ultrasound image data corresponding to the patient 102 and process the ultrasound image data to generate one or more images corresponding to the patient 102. It may be noted that the system 100 may be configured to automatically guide the clinician to a desired target location for scanning using a single acquired image. However, in certain other embodiments, more than one image may be employed to automatically guide the clinician to a desired target location for scanning.

Furthermore, in one example, the acquired image may include a two-dimensional (2D) B-mode ultrasound image. Also, in certain embodiments, the images may include pre-scan-converted or radio frequency (RF) ultrasound data. Additionally, the 2D images may include static 2D images or cine loops that include a series of 2D images or image frames acquired over time. It may be noted that although the present specification is described in terms of 2D ultrasound images, use of the present specification with three-dimensional (3D) ultrasound images and four-dimensional (4D) ultrasound images is also envisaged.

In the present specification, the object of interest is the right kidney of the patient 102. It may be noted that although the present specification is described with reference to the right kidney as the object of interest, use of the present specification for imaging anatomical regions of interest in other objects of interest is also envisaged.

In a presently contemplated configuration, the system 100 may be configured to acquire image data representative of the patient 102. In one embodiment, the system 100 may acquire image data corresponding to the patient 102 via the image acquisition device 104. Also, in one embodiment, the image acquisition device 104 may include a probe, where the probe may include an invasive probe, or a non-invasive or external probe, such as an external ultrasound probe, that is configured to aid in the acquisition of image data. Also, in certain other embodiments, image data may be acquired via one or more sensors (not shown) that may be disposed on the patient 102. By way of example, the sensors may include physiological sensors (not shown) such as positional sensors. In certain embodiments, the positional sensors may include electromagnetic field sensors or inertial sensors. These sensors may be operationally coupled to a data acquisition device, such as an imaging system, via leads (not shown), for example.

Furthermore, the ultrasound system 100 may also include a position sensing unit 106 that is operatively coupled to the ultrasound probe 104. The position sensing unit 106 may include an optical tracking system, magnetic position sensing system, a sensor in a probe holder, a motion sensing system, a laser, a camera, an electromagnetic position sensing system and/or any suitable system or combinations of systems configured to detect, in real-time, the position of the ultrasound probe 104. In some embodiments, the position sensing unit 106 may provide the probe position data to the processing subsystem 112 of the ultrasound system 100 for association with ultrasound image data acquired by the ultrasound probe 104 at the corresponding probe positions. In certain embodiments, the ultrasound probe 104 may be operable to acquire ultrasound image data covering at least a substantial portion of an organ, such as the right kidney or any suitable organ.

The system 100 may also include a medical imaging system 108 that is in operative association with the image acquisition device 104. It should be noted that although the exemplary embodiments illustrated hereinafter are described in the context of a medical imaging system, other imaging systems and applications such as industrial imaging systems and non-destructive evaluation and inspection systems, such as pipeline inspection systems, liquid reactor inspection systems, are also contemplated. Additionally, the exemplary embodiments illustrated and described hereinafter may find application in multi-modality imaging systems that employ ultrasound imaging in conjunction with other imaging modalities, position-tracking systems or other sensor systems. In one example, the multi-modality imaging system may include a positron emission tomography (PET) imaging system-ultrasound imaging system. Furthermore, in other non-limiting examples of the multi-modality imaging systems, the ultrasound imaging system may be used in conjunction with other imaging systems, such as, but not limited to, a computed tomography (CT) imaging system, a contrast enhanced ultrasound imaging system, an X-ray imaging system, an optical imaging system, a magnetic resonance (MR) imaging system and other imaging systems, in accordance with aspects of the present specification.

As noted hereinabove, in a presently contemplated configuration, the medical imaging system 108 is an ultrasound imaging system. The medical imaging system 108 may include an acquisition subsystem 110 and a processing subsystem 112, in one embodiment. Further, the acquisition subsystem 110 of the medical imaging system 108 is configured to acquire image data representative of the patient 102 via the image acquisition device 104, in one embodiment. For example, the acquired image data may include a plurality of 2D ultrasound images or slices. It may be noted that the terms images and image frames may be used interchangeably.

In addition, the acquisition subsystem 110 may also be configured to acquire images stored in the optical data storage article. It may be noted that the optical data storage article may be an optical storage medium, such as a compact disc (CD), a digital versatile disc (DVD), multi-layer structures; such as DVD-5 or DVD-9, multi-sided structures, such as DVD-10 or DVD-18, a high definition digital versatile disc (HD-DVD), a Blu-ray disc, a near field optical storage disc, a holographic storage medium, or another like volumetric optical storage medium, such as, for example, two-photon or multi-photon absorption storage format. Further, the 2D images so acquired by the acquisition subsystem 110 may be stored locally on the medical imaging system 108 in the data repository 116, for example.

Additionally, the image data acquired from the patient 102 may then be processed by the processing subsystem 112. Further, the processing subsystem 112 is also configured to receive the probe position data from the position sensing unit 106 and associate the probe position data with the acquired ultrasound image data acquired by the ultrasound probe 104 at the corresponding probe positions.

The processing subsystem 112, for example, may include one or more application-specific processors, graphical processing units, digital signal processors, microcomputers, microcontrollers, Application Specific Integrated Circuits (ASICs), Field Programmable Gate Arrays (FPGAs), Programmable Logic Arrays (PLAs), and/or other suitable processing devices. Alternatively, the processing subsystem 112 may be configured to store the acquired image data and/or the user input in a data repository 116 for later use. In one embodiment, the data repository 116, for example, may include a hard disk drive, a floppy disk drive, a compact disk-read/write (CD-R/W) drive, a Digital Versatile Disc (DVD) drive, a flash drive, and/or a solid-state storage device.

According to aspects of the present specification, the image data acquired and/or processed by the medical imaging system 108 may be employed to aid a clinician, irrespective of skill level, to arrive at the right scan plane, make automated measurements, and provide a diagnosis based on the acquired image. In certain embodiments, the processing subsystem 112 may be further coupled to a storage system, such as the data repository 116, where the data repository 116 is configured to store the acquired image data. In certain embodiments, the data repository 116 may include a local database.

Furthermore, in accordance with exemplary aspects of the present specification, the processing subsystem 112 includes a navigation platform 114 that is configured to aid in the automated determination of the right scan plane to enable automated measurements corresponding to the patient 102. The exemplary system 100 that includes the navigation platform 114 provides a fully automated framework for acquiring relevant images for clinical diagnosis, which will in turn enable the clinicians with varying skill levels to be effective in ultrasonography, thereby simplifying the workflow and enhancing the productivity of a skilled and/or less-experienced clinician. Consequently, the exemplary navigation platform 114 is configured to provide an objective, “operator independent” navigation guidance to the clinician.

Also, in the presently contemplated configuration illustrated in FIG. 1, the processing subsystem 112 is shown as including the navigation platform 114. However, in certain embodiments, the navigation platform 114 may also be used as a standalone unit that is physically separate from the processing subsystem 112 and the medical imaging system 108. By way of example, the navigation platform 114 may be external to and operatively coupled to the medical imaging system 108.

As noted hereinabove, clinically relevant outcomes depend on the skill level of the clinician in ultrasound scanning. Low-skilled operators/clinicians often struggle to identify the anatomy, which is to be followed by tasks of greater complexity, such as the identification of the right scan plane and biometry. Traditionally, the clinician positions the ultrasound probe 104 on the body of the patient 102 and moves the ultrasound probe 101 till a desired scan plane is identified. Unfortunately, this is a time consuming and laborious task, especially for low-skilled operators. Also, the outcome is very subjective and is dependent on the skill of the clinician.

The exemplary navigation platform 114 is configured to circumvent the shortcomings of the presently available techniques. More particularly, the navigation platform 114 is configured to guide and/or help the clinician navigate the ultrasound probe 104 during the imaging session, irrespective of skill level, to arrive at the right scan plane, make automated measurements, and provide a diagnosis. Specifically, the navigation platform 114 is configured to provide actionable instructions in terms of movements of the ultrasound probe 104 which help the clinician arrive at the clinical gold standard, thereby leading to consistent outcomes.

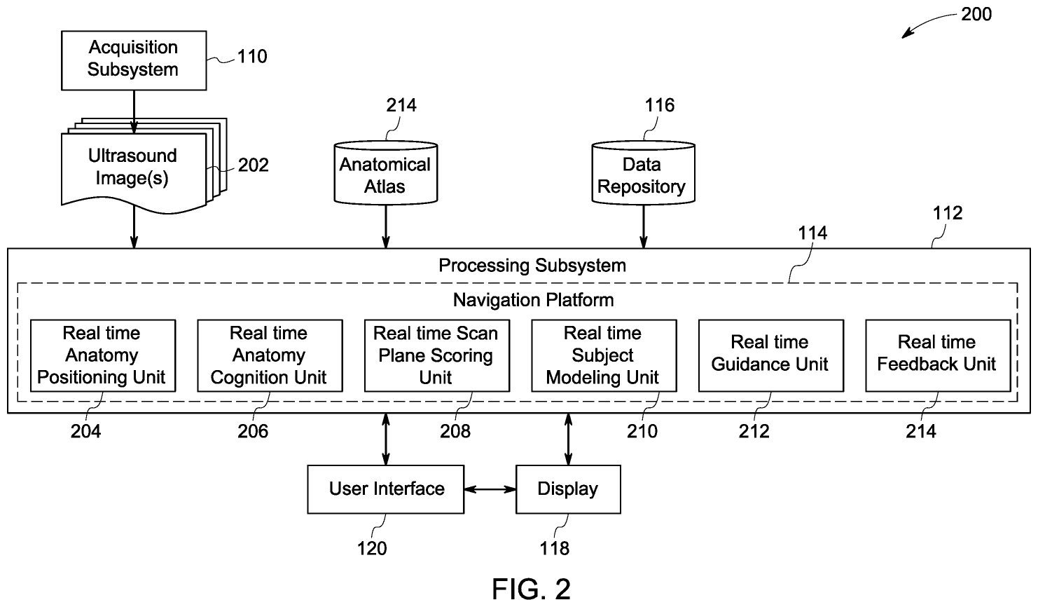

To that end, the navigation platform 114 is configured to process the acquired ultrasound image to identify, in real-time, one or more anatomical regions of interest in the patient 102. Prior to identifying the anatomical regions of interest in the patient 102, it is desirable to acquire image data corresponding to the patient 102. Accordingly, the clinician may position the ultrasound probe 104 at a determined location on the body of the patient 102 and acquire at least one image of the patient 102 via the ultrasound probe 104. Further, the navigation platform 114 is configured to determine in real-time, a current position of the ultrasound probe 104 on a body surface of patient 102 based on the acquired image and identify in real-time, one or more anatomical regions of interest in the acquired image. Additionally, the navigation platform 111 is configured to quantify in real-time, the acquired image to determine suitability of that image to one or more scan planes corresponding to a determined clinical protocol and generate in real-time, a personalized anatomical model of the patient 102 based on a current position of the ultrasound probe, the identified anatomical region(s) of interest, and the quantification of the acquired image. The navigation platform 114 is also configured to compute in real-time, a desired trajectory of the ultrasound probe 104 from the current location to a target location based on the determined clinical protocol and communicate in real-time, a desired movement of the ultrasound probe 104 based on the computed trajectory. Once the desired movement of the ultrasound probe 104 is determined, the ultrasound probe 104 may be moved along the computed trajectory based on the communicated desired movement to acquire images of the patient 102, where the acquired images include a desired anatomical region of interest. These images may be used to make measurements, diagnose a condition, suggest a treatment plan, study the efficacy of a currently ongoing treatment plan, and the like. In one example, the navigation platform 114 may be configured to access the 2D images from the local database 116. Alternatively, the 2D images may be obtained by the acquisition subsystem 110 from an archival site, a database, or an optical data storage article. Alternatively, the 2D images may be obtained by the acquisition subsystem 110 from an archival site, a database, or an optical data storage article. The working of the navigation platform 114 will be described in greater detail with reference to FIGS. 2-4.

Moreover, as illustrated in FIG. 1, the medical imaging system 108 may include a display 118 and a user interface 120. In certain embodiments, such as in a touch screen, the display 118 and the user interface 120 may overlap. Also, in some embodiments, the display 118 and the user interface 120 may include a common area. In accordance with aspects of the present specification, the display 118 of the medical imaging system 108 may be configured to display an image generated by the medical imaging system 108 based on the acquired image data. Additionally, the current location of the ultrasound probe 104, previous locations of the ultrasound probe 106, the quantification of the image, and the desired trajectory of the ultrasound probe 104 may also be visualized the display 118, Moreover, any quality metrics/indicators generated by the navigation platform 114 may also be visualized on the display 118. In one embodiment, the indicator that is representative of the quality metric may be overlaid on the corresponding image visualized on the display 118. For example, the generated indicator may be overlaid on or about the image visualized on the display 118.

In addition, the user interface 120 of the medical imaging system 108 may include a human interface device (not shown) configured to aid the clinician in manipulating image data displayed on the display 118. The human interface device may include a mouse-type device, a trackball, a joystick, a stylus, or a touch screen configured to facilitate the clinician to identify the one or more regions of interest in the images. However, as will be appreciated, other human interface devices, such as, but not limited to, a touch screen, may also be employed. Furthermore, in accordance with aspects of the present specification, the user interface 120 may be configured to aid the clinician in navigating through the images acquired by the medical imaging system 108. Additionally, the user interface 120 may also be configured to aid in manipulating and/or organizing the displayed images and/or generated indicators displayed on the display 118.

Turning now to FIG. 2, a block diagram 200 of one embodiment of the diagnostic system 100 of FIG. 1 is depicted. FIG. 2 is described with reference to the components of FIG. 1.

As previously noted with reference to FIG. 1, the acquisition subsystem 108 (see FIG. 1) is configured to aid in the acquisition of image data corresponding to an anatomical region of the patient 102 such as the right kidney. Accordingly, at least one image representative of the patient 102 may be acquired by the acquisition subsystem 110. In certain embodiments, the image may include an ultrasound image 202. It may be noted that the ultrasound image 202 may be representative of the anatomical region in the patient 102. For instance, in the example illustrated in FIG. 2, the ultrasound image 202 may include image data representative of the abdominal region of the patient 102 that includes the right kidney. As previously noted, the ultrasound image 202 may include 2D ultrasound image frames or cine loops, where the cine loops include 2D image frames acquired over time t.

Furthermore, the image data acquired by the acquisition subsystem 110 may be stored in the data repository 116. In certain embodiments, the data repository 116 may include a local database. The navigation platform 114 may be configured to access the images, such as the ultrasound images 202, from the local database 116, Alternatively, the ultrasound images 202 may be obtained by the acquisition subsystem 110 from an archival site, a database, or an optical data storage article. Moreover, in certain embodiments, the ultrasound images 202 so acquired by the acquisition subsystem 110 may be stored locally on the medical imaging system 108. By way of example, the ultrasound images 202 may be stored in the data repository 116.

Also, in the embodiments illustrated in FIGS. 1-2, the processing subsystem 110 is shown as including the navigation platform 114, where the navigation platform 114 is configured to guide and/or help the clinician navigate the ultrasound probe 104 during the imaging session, irrespective of skill level, to arrive at the right scan plane, make automated measurements, and provide a diagnosis. Specifically, the navigation platform 114 is configured to provide actionable instructions in terms of movements of the ultrasound probe 104 which help the clinician arrive at the clinical gold standard, thereby leading to consistent outcomes. Moreover, as previously noted in certain embodiments, the navigation platform 114 may also be used as a standalone unit that is physically separate from the processing subsystem 110 and the medical imaging system 108.

In one embodiment, the navigation platform 114 may include a real-time anatomy positioning unit 204, a real-time anatomy cognition unit 206, a real-time scan plane scoring unit 208, a real-time subject modeling unit 210, a real-time guidance unit 212, and a real-time feedback unit 214. It may be noted that although the configuration of FIG. 2 depicts the navigation platform 114 as including the anatomy positioning unit 204, the anatomy cognition unit 206, the scan plane scoring unit 208, the subject modeling unit 210, the guidance unit 212, and the feedback unit 214, fewer or greater number of such units may be used.

In accordance with aspects of the present specification, the navigation platform 114 is configured to provide assistance to the clinician in locating the desired anatomical region of interest. In the present example, the desired anatomical region of interest is the right kidney of the patient 112. Accordingly, the clinician may position the ultrasound probe 104 on the surface of the body of the patient 102 to image the patient 102, Subsequently, the navigation platform 114, via use of a deep learning algorithm, is configured to detect anatomical regions seen in the acquired images 202 and readjust internal anatomy in an anatomical atlas based on detected anatomical regions. By way of example, the location of the right kidney in the anatomical atlas is adjusted to match the patient 102.

The anatomy positioning unit 204 is configured to track, in real-time, a spatial location of the ultrasound probe 104 and situate the current location of the ultrasound probe 104 within the anatomy of the patient 102. Accordingly, in one embodiment, the anatomy positioning unit 204 employs an anatomical atlas to provide an anatomical context. The anatomical atlas may be retrieved from a database such as an anatomical atlas repository 214, in certain embodiments. Further, the anatomy positioning unit 204 is configured to make the anatomical atlas “subject” or “patient” specific by deforming the anatomical atlas to match external landmarks of the patient 102. Accordingly, one or more anatomical landmarks such as the ribs, solar plexus, and the like on the body surface of the patient 102 are identified. In certain embodiments, the clinician may manually identify the external bony landmarks on the body surface of the patient 102. These landmarks may be used to align the anatomical atlas to the patient 102. As will be appreciated, in certain situations, the external alignment however may not guarantee accurate alignment of the internal anatomy of the patient 102. Accordingly, the system 200 and the navigation platform 114 in particular is configured to instruct the clinician is instructed to perform a “scout sweep” around the anatomical region of interest in the anatomical atlas.

Moreover, the anatomy positioning unit 204 is configured to automatically identify one or more landmarks on the body surface of the patient 102. In one example, six (6) landmarks may be automatically identified on the body surface of the patient 102 via use of a camera. Further, the anatomy positioning unit 201 is configured to register the body surface of the patient 102 with the anatomical atlas based on the identified external landmarks to generate an exterior of a personalized anatomical model of the patient 102, Furthermore, one or more position sensors (not shown in FIG. 2) may be used to determine the current spatial location or position of the ultrasound probe 104 based on the acquired ultrasound image 202. In one example, the position sensing unit 106 may be used to obtain the current spatial location of the ultrasound probe 104. The position sensing unit 106 may be employed to capture the (x, y, z) coordinates of the current location of the ultrasound probe 104 and the yaw, pitch and roll orientations of the ultrasound probe 104. Further, these coordinates and orientations of the ultrasound probe 104 are mapped to the exterior of the personalized anatomical model of the patient 102. This mapping aids in identifying the current location of the ultrasound probe 104 based on the personalized anatomical model of the patient 102. This mapping also aids the clinician in viewing potential locations of the internal organ(s) of the patient 102 with reference to the current probe location on the ultrasound image 202.

It may be noted that consequent to the processing via the anatomy positioning unit 201, a visual, anatomical context of the current location of the ultrasound probe 104 is generated. In particular, the anatomy positioning unit 204 is configured to “inform” the clinician as to “where” the ultrasound probe 104 is currently positioned on the body surface of the patient 102, This anatomical context may be used to aid in guiding the clinician on the anatomical atlas to a desired anatomical location in the patient 102.

Furthermore, the real-time anatomy cognition unit 206 is configured to identify and localize, in real-time, anatomies or anatomical regions that are present in the acquired ultrasound image 202. In particular, the anatomy cognition unit 206 is configured to detect all visible organs in the ultrasound image 202. In certain embodiments, the anatomical cognition unit 206 employs a deep learning technique to identify the anatomies in the ultrasound image 202. In certain embodiments, pre-trained neural networks may be used detect the anatomies in the ultrasound image 202. Additionally, the anatomy cognition unit 206 is configured to generate a bounding box corresponding to each of the detected anatomies. The bounding boxes are configured to encompass a corresponding detected anatomy.

In accordance with further aspects of the present specification, the anatomy cognition unit 206 is further configured to continuously and robustly track the identified anatomies in the ultrasound image 202 across successive frames notwithstanding any changes in the presentation of the identified anatomies. Moreover, if a change in scene/shot is detected, the anatomy cognition unit 206 is configured to re-detect and re-track the anatomies.

Processing the ultrasound image 202 via the anatomy cognition unit 206 provides anatomical awareness to the clinician. In particular, while the clinician is scanning the patient 102, the anatomy cognition unit 206 provides the clinician with a visual representation of “what” (for example, the identified anatomies) is being visualized. In the present example, as the clinician is scanning the patient 102, the anatomy cognition unit 206 is configured to detect the presence of the right kidney in the ultrasound images 202.

With continuing reference to the navigation platform 114, the scan plane scoring unit 208 is configured to quantify, in real-time, the ultrasound image 202 to determine suitability of that ultrasound image 202 to one or more scan planes corresponding to a determined clinical protocol. More particularly, the scan plane scoring unit 208 is configured to quantify the ultrasound image 202 by rating the ultrasound image 202 based on a clinical standard to generate a proximity score corresponding to each scan plane of the one or more scan planes. In one embodiment, the scan plane scoring unit 208 may employ a deep learning technique to rate the ultrasound image 202 to generate the proximity scores corresponding to each of the one or more scan planes.

By way of example, following the scanning of the patient 102 by the clinician, one or more images of the ultrasound images 202 that include the right kidney are identified. The scan plane scoring unit 208 is configured to process scan planes associated with each of these identified images to determine the suitability of the scan planes for measurements and/or diagnosis.

Subsequent to the generation of the proximity scores, the real-time subject modeling unit 210 is configured to generate, in real-time, a personalized anatomical model of the patient 102 based on the current position of the ultrasound probe 104, the identified anatomical regions of interest, the bounding boxes that encompass the identified anatomical regions of interest, and the quantification of the ultrasound image 202. By way of example, the subject modeling unit 210 is configured to build an anatomical twin of the right kidney of the patient 102. It may be noted that in one embodiment, the personalized anatomical model may be in the same coordinate system as the exterior of the personalized anatomical model. Further, in some embodiments, an interior of the personalized anatomical model of the patient 102 may be updated based on one or more of the personalized anatomical model, the bounding boxes corresponding to the one or more detected anatomical regions of interest, and the proximity scores corresponding to the one or more scan planes. Accordingly, the subject modeling unit 210 aids the system 100 in continuously “learning” the anatomical layout of the patient 102 based on the current location of the ultrasound probe 104 and the identified anatomical regions of interest. In particular, the subject modeling unit 210 is configured to customize the personalized anatomical model as the patient 102 is scanned. Additionally, subsequent to the scoring of the scan planes, the subject modeling unit 210 is configured to identify an optimal scan plane from the one or more scan planes for imaging the right kidney of the patient 102.

Once the current location of the ultrasound probe 104 is identified and the personalized anatomical model is generated and/or updated, it is desirable to “guide” the clinician from the current location of the ultrasound probe 104 to a “desired” or “target” location to enable accurate imaging of the desired anatomical region of interest in the patient 102. Accordingly, the real-time guidance unit 212 is configured to compute, in real-time, a desired trajectory of the ultrasound probe 104 from the current location of the ultrasound probe 104 to the target location based on the determined clinical protocol. In certain embodiments, the guidance unit 212 is configured to compute the desired trajectory of the ultrasound probe 104 by charting a path on the body surface of the patient 102 from the current position of the ultrasound probe 104 to the target location based on the current position of the ultrasound probe 104, the bounding boxes corresponding to the one or more detected anatomical regions of interest, the identified anatomical regions of interest, or combinations thereof. More particularly, in certain embodiments, to acquire the long axis of the right kidney, points corresponding to the interior of the personalized anatomical model are circumscribed by an ellipsoid. In addition, a plane passing through the major axis of the ellipsoid is identified. Further, an orientation of the identified plane is computed. The desired trajectory is then computed from the current location of the ultrasound probe 104 to the location of the identified plane. In one example, the desired trajectory may be determined based on minimum manifold distance between the current location of the ultrasound probe 104 and the location of the identified plane. Subsequently, one or more ultrasound images around this identified plane are acquired and scored to determine the optimal scan plane.

As previously noted, the system 100 and the navigation platform 114 in particular is configured to provide actionable instructions, in real-time, to the clinician based on the computed trajectory from the current location of the ultrasound probe 104 to the target location on the patient 102. Accordingly, the real-time feedback unit 214 is configured to compute and communicate, in real-time, suggested probe movements to the clinician to reach the target location from the current probe location based on the determined clinical protocol, in the present example, the feedback unit 214 is configured to compute and communicate desired movements of the ultrasound probe 104 to the clinician to navigate the ultrasound probe 104 from the current location of the ultrasound probe 104 to the target location to enable imaging the right kidney of the patient 102. In particular, during the real-time scanning of the patient 102, the feedback unit 214 is configured to provide feedback to the clinician to “guide” the clinician to an optimal scan plane for imaging the right kidney of the patient 102.

Accordingly, the clinician may move the ultrasound probe 104 along the computed trajectory to the target location based on the feedback/guidance received from the feedback unit 214. In certain embodiments, the feedback unit 214 may be configured to communicate the desired movement of the ultrasound probe 104 to the clinician via a real-time indicator. By way of example, the feedback unit 214 is configured to provide the real-time indicator to the clinician by visualizing the real-time indicator on a display such as the display 118 to guide the clinician to the target location. Additionally or alternatively, the feedback unit 214 may also play an audio-indicator of the real-time indicator, in real-time, to guide the clinician to the target location. Further, the real-time indicator may include a “color” indicator or a “directional” indicator. In one embodiment, the feedback unit 214 is configured to change the color of the ultrasound probe 104 to indicate that the desired anatomical region has been located. By way of example, if the right kidney is identified in a given ultrasound image 202, the color of the ultrasound probe 104 may be changed to green to indicate the identification of the right kidney in the ultrasound image 202. Once the ultrasound probe 104 is situated at the target location, the clinician may capture one or more desired images, where the desired images include the desired anatomical region(s) of interest.

Furthermore, subsequent to the acquisition of the desired images, the acquired/captured images may be visualized, in real-time, on the display 118. Additionally, the captured ultrasound image(s), one or more of the bounding boxes, the computed trajectory, the quantification of the ultrasound image 202, and the like may also be visualized on the display 118. In certain embodiments, the bounding boxes, the computed trajectory, and/or the quantification of the ultrasound image 202 may be superimposed on the ultrasound image 202.

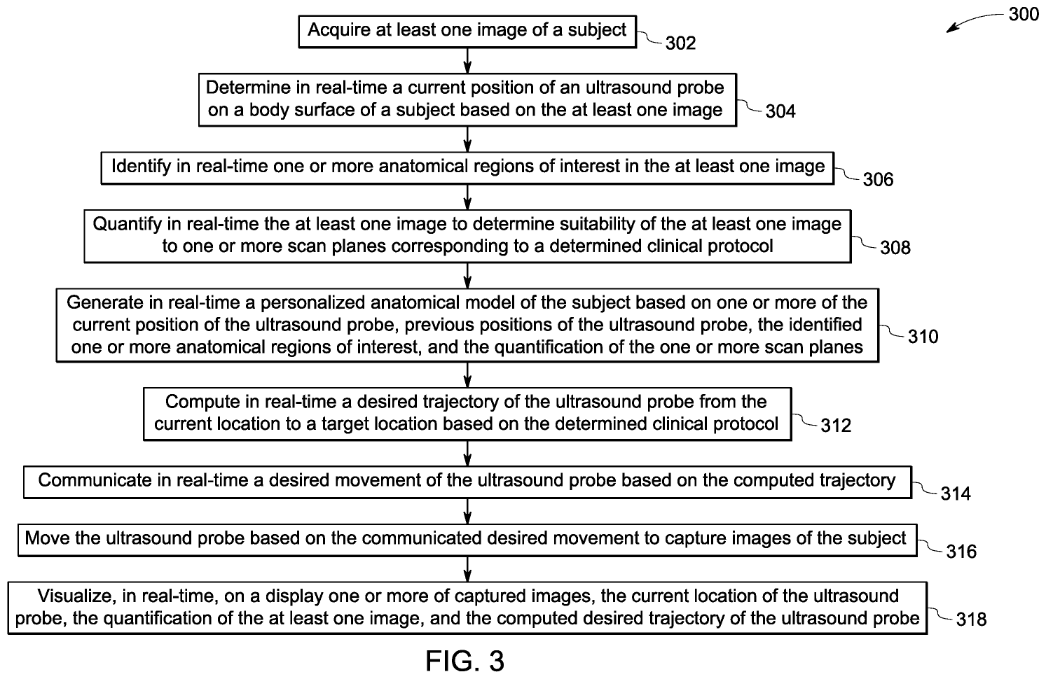

Turning now to FIG. 3, a flow chart of exemplary logic 300 for a method for ultrasound imaging is illustrated. It may be noted that method 300 provides an objective, “operator independent” navigation guidance to the clinician to perform the imaging of the patient 102. In the present specification, embodiments of the exemplary method 300 of FIG. 3 may be described in a general context of computer executable instructions on a computing system or a processor. Generally, computer executable instructions may include routines, programs, objects, components, data structures, procedures, modules, functions, and the like that perform particular functions or implement particular abstract data types.

Additionally, embodiments of the exemplary method 300 of FIG. 3 may also be practised in a distributed computing environment where optimization functions are performed by remote processing devices that are linked through a wired and/or wireless communication network. In the distributed computing environment, the computer executable instructions may be located in both local and remote computer storage media, including memory storage devices.

Further, in FIG. 3, the method 300 for ultrasound imaging is illustrated as a collection of blocks in a logical flow chart, which represents operations that may be implemented in hardware, software, or combinations thereof. The various operations are depicted in the blocks to illustrate the functions that are performed. In the context of software, the blocks represent computer instructions that, when executed by one or more processing subsystems, perform the recited operations.

The order in which the method 300 of FIG. 3 is described is not intended to be construed as a limitation, and any number of the described blocks may be combined in any order to implement the exemplary methods disclosed herein, or equivalent alternative methods. Additionally, certain blocks may be deleted from the exemplary methods or augmented by additional blocks with added functionality without departing from the spirit and scope of the subject matter described herein. Although, the exemplary embodiments illustrated hereinafter are described in the context of a medical imaging system, it will be appreciated that use of the systems and methods in industrial applications is also contemplated in conjunction with the present specification.

The working of the system 100 (see FIG. 1) and the navigation platform 114 (see FIG. 1) in particular may be better understood with reference to the exemplary logic depicted in FIG. 3. In accordance with exemplary aspects of the present specification, the method of imaging 300 provides actionable instructions in terms of probe movements to aid the clinician in consistently arriving at the clinical gold standard, thereby leading to consistent outcomes is presented, More particularly, the method of imaging 300 guides and/or helps the clinician in navigating the ultrasound probe 104 during the imaging session, irrespective of skill level, to arrive at the right scan plane, make automated measurements, and formulate a diagnosis. Also, in certain embodiments, the navigation platform 114 may be employed to perform the steps of the method 300. The method 300 is describe with respect to the components of FIGS. 1-2.

The method 300 starts at step 302, where the clinician may position the ultrasound probe 104 on the surface of the body of the patient 102 to image the patient 102. At least one ultrasound image may be acquired, as indicated by step 302. Further, a current location of the ultrasound probe 104 on the body surface of the patient 102 may be determined in real-time. An anatomical atlas may be used to provide an anatomical context. A device such as a camera may be used to identify one or more external landmarks on the body surface of the patient 102. In some examples, six (6) landmarks may be identified on the body surface of the patient 102. Also, the anatomical atlas is customized and made “subject” or “patient” specific by deforming the anatomical atlas to match the external landmarks of the patient 102. In one embodiment, the body surface of the patient 102 is registered with the anatomical atlas based on the identified external landmarks to generate an exterior of a personalized anatomical model of the patient 102. Furthermore, the current spatial location or position of the ultrasound probe 101 is determined based on the acquired ultrasound image 202 via use of one or more position sensors or the position sensing unit 106. In particular, (x, y, z) coordinates of the current location of the ultrasound probe 104 and the yaw, pitch and roll orientations of the ultrasound probe 104 are determined and are mapped to the exterior of the personalized anatomical model of the patient 102 to identify the current location of the ultrasound probe 104 based on the personalized anatomical model of the patient 102.

Subsequently, at step 306, anatomies or anatomical regions that are present in the acquired ultrasound image 202 are identified and localized, in real-time. In particular, all visible organs in the ultrasound image 202 are detected. In certain embodiments, a deep learning technique is used to identify the anatomies in the ultrasound image 202, Furthermore, a bounding box corresponding to each of the detected anatomies in the ultrasound image is generated. The bounding boxes are configured to encompass a corresponding detected anatomy. Additionally, at step 306, the identified anatomies in the ultrasound image 202 may be continuously tracked across successive frames. If a change in scene/shot is detected, the anatomies are re-detected and re-tracked.

Furthermore, at step 308, the ultrasound image 202 is quantified, in real-time, to determine suitability of that ultrasound image 202 to one or more scan planes corresponding to a determined clinical protocol. Accordingly, in one embodiment, the ultrasound image 202 may be rated based on a clinical standard to generate a proximity score corresponding to each scan plane of the one or more scan planes. Also, in some embodiments, a deep learning technique may be used to perform the rating of the ultrasound image to generate the proximity scores corresponding to each of the one or more scan planes.

Moreover, a personalized anatomical model of the patient 102 is generated, in real-time, based on the current position of the ultrasound probe 104, the identified anatomical regions of interest, the bounding boxes that encompass the identified anatomical regions of interest, and the quantification of the ultrasound image 202, as indicated by step 310. Additionally, in certain embodiments, an interior of the personalized anatomical model of the patient 102 may be updated based on one or more of the personalized anatomical model, the bounding boxes corresponding to the one or more detected anatomical regions of interest, and the proximity scores corresponding to the one or more scan planes.

In addition, subsequent to the identification of the current location of the ultrasound probe 104, the clinician is guided to traverse a path from the current location of the ultrasound probe 104 to a “desired” or “target” location to enable accurate imaging of a desired anatomical region of interest in the patient 102, as indicated by step 312. Accordingly, a desired trajectory of the ultrasound probe 104 from the current location to the target location is determined, in real-time, based on the determined clinical protocol. In certain embodiments, the desired trajectory is computed by charting a path on the body surface of the patient 102 from the current position of the ultrasound probe 104 to the target location based on the current position of the ultrasound probe 104, the bounding boxes corresponding to the one or more detected anatomical regions of interest, the identified anatomical regions of interest, or combinations thereof. By way of example, to acquire the long axis of the right kidney, points corresponding to the interior of the personalized anatomical model are circumscribed by an ellipsoid and an orientation of a plane passing through the major axis (i.e., identified plane) of the ellipsoid is determined. The desired trajectory is computed from the current location of the ultrasound probe 104 to the location of the identified plane. In one example, the desired trajectory may be determined based on minimum manifold distance between the current location of the ultrasound probe 104 and the location of the identified plane. Further, one or more ultrasound images around this identified plane are acquired and scored to determine the optimal scan plane.

As previously noted, actionable instructions are provided in real-time to the clinician based on the computed trajectory from the current location of the ultrasound probe 104 to the target location on the patient 102. Accordingly, at step 314, desired suggested probe movements are computed and communicated, in real-time, to the clinician to arrive at the target location from the current probe location based on the determined clinical protocol.

The actionable instructions so generated are communicated to the clinician to guide the clinician to the target location, as depicted by step 316. Accordingly, the clinician may move the ultrasound probe 104 to the target location based on the feedback/guidance received. In certain embodiments, the desired movement of the ultrasound probe 104 may be communicated to the clinician via a real-time indicator. By way of example, the may include a visualization of the real-time indicator on a display such as the display 118 and/or playing an audio-indicator, in real-time, to guide the clinician to the target location. Once the ultrasound probe 104 is situated at the target location, the clinician may capture one or more desired images, where the desired images include the desired anatomical region(s) of interest.

Further, at step 318, the captured images may be visualized, in real-time, on the display 118. Additionally, the captured images, the ultrasound image 202, one or more of the bounding boxes, the computed trajectory, the quantification of the ultrasound image 202, and the like may also be visualized on the display 118. In certain embodiments, the bounding boxes, the computed trajectory, and/or the quantification of the ultrasound image 202 may be superimposed on the ultrasound image 202.

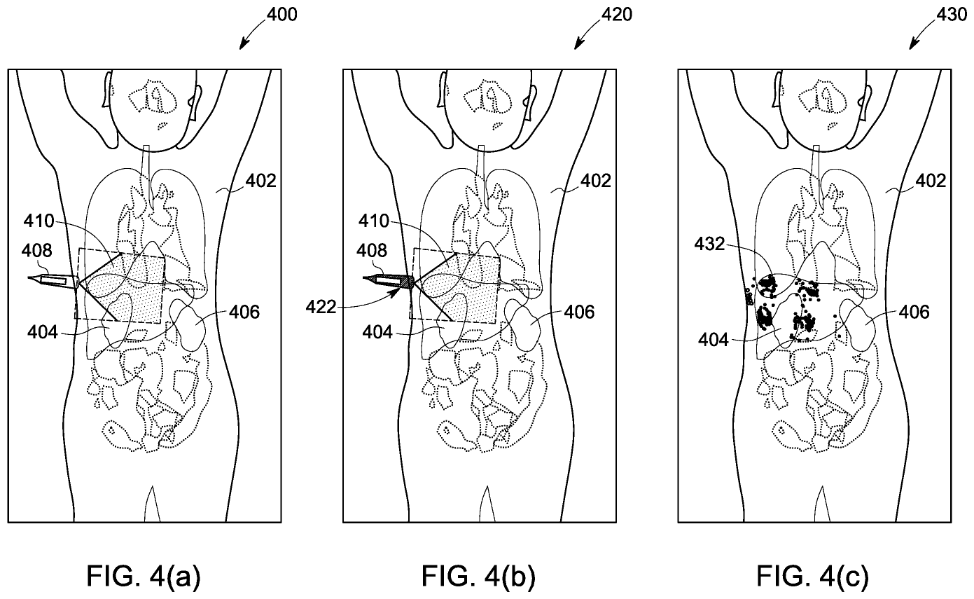

FIGS. 4(a)-4(c) depict a diagrammatic illustration of one example of the exemplary method for ultrasound imaging 300 of FIG. 3, in accordance with aspects of the present specification. Also, FIGS. 4(a)-4(c) are described with reference to FIGS. 1-3.

FIG. 4(a) depicts a diagrammatical illustration 400 of a patient 402 (such as the patient 102) being imaged. Reference numeral 404 is used to represent a desired anatomical region of interest such as the right kidney of the patient 402, The left kidney of the patient 402 is represented by reference numeral 406. Also, reference numeral 408 depicts an ultrasound probe such as the ultrasound probe 104, while a scan plane is represented by reference numeral 410. In FIG. 4(a), a tracking of a current location of the ultrasound probe 408 is depicted. In one embodiment, an electromagnetic sensor in the position sensing unit 106 may be used to track the location of the ultrasound probe 408. Furthermore, in FIG. 4(a), the ultrasound probe 408 is depicted as being position near the lower end of the ribs of the patient 402.

Turning now to FIG. 4(b), a diagrammatical illustration 420 of one example of real-time deep learning of anatomy of the patient 402 is depicted. In particular, as the patient 402 is being scanned, the navigation platform 114 is configured to continue to “learn,” via use of a deep learning technique, the anatomy of the patient 402. In addition, one example of communication of a real-time indicator 422 the clinician is also depicted. By way of example, as the clinician scans the patient 402, if the right kidney is detected in an image, the system 100 is configured to communicate the real-time indicator to the clinician, where the real-time indicator 422 is configured to inform the clinician of the detected anatomical region of interest in a given image. In one particular example, the color of the ultrasound probe 408 may be changed to green to indicate that the right kidney has been identified in the ultrasound image. However, as previously noted, other types of real-time indicators such as a visual indicator and/or an audio-indicator of the visual indicator may also be used to guide the clinician to the target location.

Referring to FIG. 4(c), a diagrammatical illustration 430 of real-time patient specific model building is depicted. This personalized anatomical model may then be used to guide the clinician to image and examine the right kidney and make any required measurements. Reference numeral 432 is generally representative of bounding boxes corresponding to the images that include the right kidney 404. In particular, in FIG. 4(c), a patient specific alignment of the anatomical region of interest, such as the right kidney 404 is depicted.

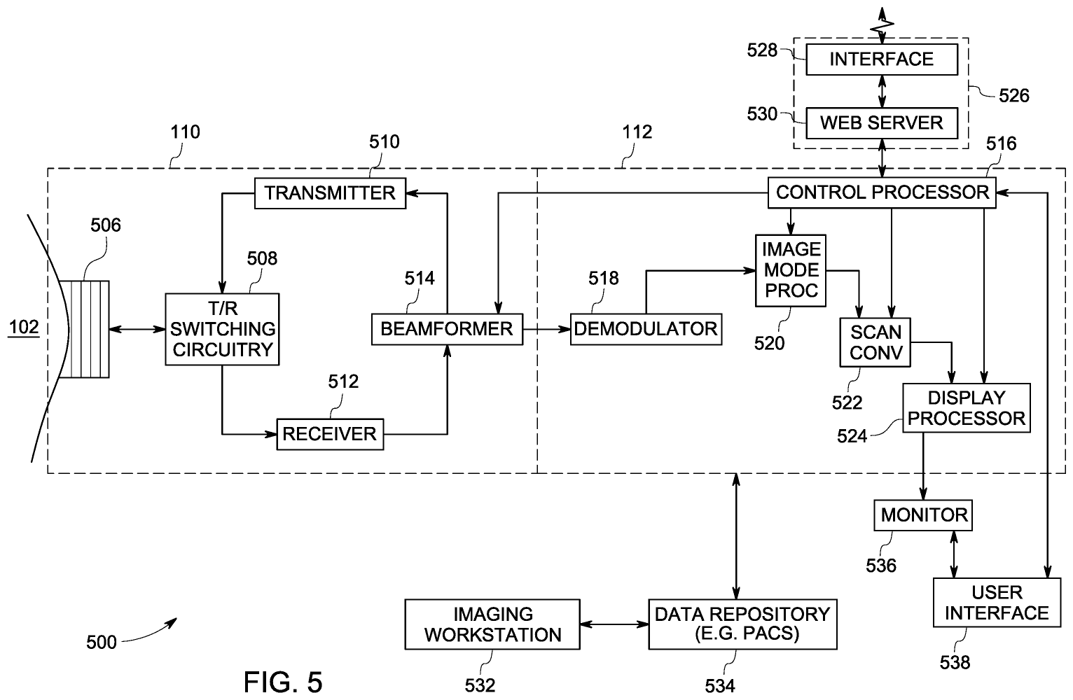

As previously noted with reference to FIG. 1, the medical imaging system 108 may include an ultrasound imaging system. FIG. 5 is a block diagram of an embodiment of an ultrasound imaging system 500 depicted in FIG. 1. The ultrasound system 500 includes an acquisition subsystem, such as the acquisition subsystem 112 of FIG. 1 and a processing subsystem, such as the processing subsystem 112 of FIG. 1. The acquisition subsystem 112 may include a transducer assembly 506. In addition, the acquisition subsystem 110 includes transmit/receive switching circuitry 508, a transmitter 510, a receiver 512, and a beamformer 514. It may be noted that in certain embodiments, the transducer assembly 506 is disposed in the probe 104 (see FIG. 1). Also, in certain embodiments, the transducer assembly 506 may include a plurality of transducer elements (not shown) arranged in a spaced relationship to form a transducer array, such as a one-dimensional or two-dimensional transducer array, for example. Additionally, the transducer assembly 506 may include an interconnect structure (not shown) configured to facilitate operatively coupling the transducer array to an external device (not shown), such as, but not limited to, a cable assembly or associated electronics. In the illustrated embodiment, the interconnect structure may be configured to couple the transducer array to the T/R switching circuitry 508.

The processing subsystem 112 includes a control processor 516, a demodulator 518, an imaging mode processor 520, a scan converter 522 and a display processor 524. The display processor 524 is further coupled to a display monitor 536, such as the display 116 (see FIG. 1), for displaying images. User interface 538, such as the user interface area 118 (see FIG. 1), interacts with the control processor 516 and the display monitor 536. The control processor 516 may also be coupled to a remote connectivity subsystem 526 including a remote connectivity interface 528 and a web server 530. The processing subsystem 112 may be further coupled to a data repository 532, such as the data repository 114 of FIG. 1, configured to receive and/or store ultrasound image data. The data repository 532 interacts with an imaging workstation 534.

The aforementioned components may be dedicated hardware elements such as circuit boards with digital signal processors or may be software running on a general-purpose computer or processor such as a commercial, off-the-shelf personal computer (PC). The various components may be combined or separated according to various embodiments of the invention. Thus, those skilled in the art will appreciate that the present ultrasound imaging system 500 is provided by way of example, and the present specifications are in no way limited by the specific system configuration.

In the acquisition subsystem 110, the transducer assembly 506 is in contact with the patient 102. The transducer assembly 506 is coupled to the transmit/receive (T/R) switching circuitry 508. Also, the T/R switching circuitry 508 is in operative association with an output of transmitter 510 and an input of the receiver 512. The output of the receiver 512 is an input to the beamformer 514. In addition, the beamformer 514 is further coupled to the input of the transmitter 510 and to the input of the demodulator 518. The beamformer 514 is also operatively coupled to the control processor 516 as shown in FIG. 5.

In the processing subsystem 112, the output of demodulator 518 is in operative association with an input of the imaging mode processor 520. Additionally, the control processor 516 interfaces with the imaging mode processor 520, the scan converter 522 and the display processor 524. An output of imaging mode processor 520 is coupled to an input of scan converter 522. Also, an output of the scan converter 522 is operatively coupled to an input of the display processor 524. The Output of display processor 524 is coupled to the monitor 536.

Furthermore, the foregoing examples, demonstrations, and process steps such as those that may be performed by the system may be implemented by suitable code on a processor-based system, such as a general-purpose or special-purpose computer. It should also be noted that different implementations of the present specification may perform some or all of the steps described herein in different orders or substantially concurrently, that is, in parallel. Additionally, the functions may be implemented in a variety of programming languages, including but not limited to Ruby, Hypertext Preprocessor (PHP), Perl, Delphi, Python, C, C++, or Java. Such code may be stored or adapted for storage on one or more tangible, machine-readable media, such as on data repository chips, local or remote hard disks, optical disks (that is, CDs or DVDs), solid-state drives, or other media, which may be accessed by the processor-based system to execute the stored code. Note that the tangible media may include paper or another suitable medium upon which the instructions are printed. For instance, the instructions may be electronically captured via optical scanning of the paper or other medium, then compiled, interpreted or otherwise processed in a suitable manner if necessary, and then stored in the data repository or memory.

It may be noted that the foregoing examples, demonstrations, and process steps that may be performed by certain components of the present systems, for example by the processing subsystem 112 and the navigation platform 114 in particular, may be implemented by suitable code on a processor-based system. The processor-based system, for example, may include a general-purpose or a special-purpose computer. It may also be noted that different implementations of the present specification may perform some or all of the steps described herein in different orders or substantially concurrently.

As will be appreciated, ultrasound imaging, more than other medical imaging modalities, depends on the skill of the clinician to acquire relevant images for clinical diagnosis. The various systems and methods for ultrasound imaging described hereinabove provide a robust framework for intelligent, next generation ultrasound system that enables operators or clinicians with varying skill levels to be effective in ultrasonography. In particular, the systems and methods provide an operator independent ultrasound navigation and guidance system that helps any clinician, irrespective of skill level, to arrive at the right scan plane, make automated measurements, and formulate an appropriate diagnosis. Additionally, the methods and systems provide operator independent actionable instructions in terms of probe movements to aid the clinician in arriving at the clinical gold standard which will in turn lead to consistent outcomes, irrespective of the skill level of the sonographer. The systems and methods described hereinabove also enable clinicians with lower skill levels in resource constrained geographies to efficiently perform ultrasound imaging. Furthermore, the systems and methods may also make sonography attractive/accessible to non-traditional users such as anesthesiologists and general practitioners.

Additionally, the various systems and methods are automated and configured to provide guidance in real-time, thereby circumventing the need for excessive manual intervention. Consequently, dependency on highly trained professionals is reduced. In addition, the scan time may be dramatically minimized when compared to manual image acquisition and measurement, thereby increasing the throughput. By way of example, for rural setups with high volumes of scanning, these systems and methods aid in decreasing the net scan time, thereby enhancing handling of higher volumes.

Furthermore, the systems and methods aid in enhancing the accuracy of imaging by reducing the variability in assisting patients. Moreover, the productivity may be increased by avoiding multiple examinations and/or minimizing the need for support of expert clinicians. The easy and fast workflow derived provided by the methods and systems for ultrasound imaging may aid in enhancing the skill and utilization of midwives or paramedics across the world and also encourage adoption of ultrasound to assist labor in geographies with fewer experienced sonographers.

Although specific features of embodiments of the present specification may be shown in and/or described with respect to some drawings and not in others, this is for convenience only. It is to be understood that the described features, structures, and/or characteristics, illustrated in the figures and described herein, may be combined and/or used interchangeably in any suitable manner in the various embodiments, for example, to construct additional assemblies and methods for use in diagnostic imaging.

While only certain features of the present specification have been illustrated and described herein, many modifications and changes will occur to those skilled in the art. It is, therefore, to be understood that the appended claims are intended to cover all such modifications and changes as fall within the true spirit of the invention.

1. A method for ultrasound imaging, the method comprising:
acquiring, via an ultrasound probe, at least one image of a subject;
determining in real-time, via a navigation platform, a current position of an ultrasound probe on a body surface of a subject based on the at least one image;
identifying in real-time, via the navigation platform, one or more anatomical regions of interest in the at least one image;
quantifying in real-time, via the navigation platform, the at least one image to determine suitability of the at least one image to one or more scan planes corresponding to a determined clinical protocol;
generating in real-time, via the navigation platform, a personalized anatomical model of the subject based on a current position of the ultrasound probe, the identified one or more anatomical regions of interest, and the quantification of the at least one image;
computing in real-time, via the navigation platform, a desired trajectory of the ultrasound probe from the current location to a target location based on the determined clinical protocol;
communicating in real-time, via the navigation platform, a desired movement of the ultrasound probe based on the computed trajectory; and
moving the ultrasound probe along the computed trajectory based on the communicated desired movement to acquire images of the subject, wherein the acquired images comprise a desired anatomical region of interest.

2. The method of claim 1, wherein determining the current position of the ultrasound probe on the body surface of the subject comprises:
identifying one or more anatomical landmarks on the body surface of the subject;
registering the body surface of the subject with an anatomical atlas based on the one or more landmarks to generate an exterior of the personalized anatomical model of the subject;
obtaining, via use of one or more position sensors, positional coordinates and orientations of the ultrasound probe; and
mapping the positional coordinates and the orientations of the ultrasound probe to the exterior of the personalized anatomical model of the subject to identify the current location of the ultrasound probe based on the personalized anatomical model of the subject.

3. The method of claim 1, wherein identifying the one or more anatomical regions of interest comprises:
detecting, via use of a deep learning technique, one or more anatomical regions of interest in the at least one image of the subject; and
generating a bounding box corresponding to each of the one or more detected anatomical regions of interest, wherein each bounding box is configured to encompass a corresponding detected anatomical region of interest of the one or more anatomical regions of interest.

4. The method of claim 3, wherein quantifying the at least one image comprises rating, via a deep learning technique, the at least one image based on a clinical standard to generate a proximity score corresponding to each scan plane of the one or more scan planes.

5. The method of claim 4, farther comprising the updating an interior of the personalized anatomical model of the subject based on one or more of the personalized anatomical model, the bounding boxes corresponding to the one or more detected anatomical regions of interest, and the proximity scores corresponding to the one or more scan planes.

6. The method of claim 4, wherein computing the desired trajectory of the ultrasound probe comprises charting a path on the body surface of the subject from the current position of the ultrasound probe to the target location based on the current position of the ultrasound probe, the bounding boxes corresponding to the one or more detected anatomical regions of interest, the identified anatomical regions of interest, or combinations thereof.

7. The method of claim 6, wherein communicating in real time the desired movement of the ultrasound probe comprises providing desired movements of the ultrasound probe based on the computed trajectory to guide the user to the target location.

8. The method of claim 7, wherein communicating in real time the desired movement of the ultrasound probe comprises providing a real-time indicator to a user, wherein providing the real-time indicator comprises one or more of visualizing the real-time indicator on a display, playing an audio-indicator of the real-time indicator, and visualizing a color indicator to guide the user to the target location.

9. The method of claim 8, further comprising visualizing in real-time on a display the at least one image, the current location of the ultrasound probe, the quantification of the at least one image, and the desired trajectory of the ultrasound probe.

10. The method of claim 9, further comprising superimposing the real-time indicator on the at least one image.

11. A system, comprising:
a navigation platform comprising:
an anatomy positioning unit configured to determine in real-time a current position of an ultrasound probe on a body surface of a subject based on at least one image;
an anatomy cognition unit configured to identify in real-time one or more anatomical regions of interest in the at least one image;
a scan plane scoring unit configured to quantify in real-time the at least one image to determine suitability of the at least one image to one or more scan planes corresponding to a determined clinical protocol;
a subject modeling unit configured to generate in real-time a personalized anatomical model of the subject based on a current position of the ultrasound probe, previous positions of the ultrasound probe, the identified one or more anatomical regions of interest, and the quantification of the at least one image, or combinations thereof;
a guidance unit configured to compute in real-time a desired trajectory of the ultrasound probe from the current location to a target location based on the determined clinical protocol; and
a feedback unit configured to communicate in real-time a desired movement of the ultrasound probe based on the computed trajectory.

12. The system of claim 11, wherein to determine the current position of the ultrasound probe on the body surface of the subject, the anatomy positioning unit is configured to:
identify one or more anatomical landmarks on the body surface of the subject;
register the body surface of the subject with an anatomical atlas based on the one or more landmarks to generate an exterior of the personalized anatomical model of the subject;
obtain, via use of one or more position sensors, positional coordinates and orientations of the ultrasound probe; and
map the positional coordinates and the orientations of the ultrasound probe to the exterior of the personalized anatomical model of the subject to identify the current location of the ultrasound probe based on the personalized anatomical model of the subject.

13. The system of claim 11, wherein to identify the one or more anatomical regions of interest the anatomy cognition unit is configured to:
detect, via use of a deep learning technique, one or more anatomical regions of interest in the at least one image of the subject; and
generate a bounding box corresponding to each of the one or more detected anatomical regions of interest, wherein each bounding box is configured to encompass a corresponding detected anatomical region of interest of the one or more detected anatomical regions of interest.

14. The system of claim 13, wherein to quantify the at least one image the scan plane scoring unit is configured to rate, via a deep learning technique, the at least one image based on a clinical standard to generate a proximity score corresponding to each scan plane of the one or more scan planes.

15. The system of claim 14, wherein the subject modeling unit is configured to update an interior of the personalized anatomical model of the subject based on one or more of the personalized anatomical model, the bounding boxes corresponding to the one or more detected anatomical regions of interest, and the proximity scores corresponding to the one or more scan planes.

16. The system of claim 14, wherein to compute the desired trajectory of the ultrasound probe the guidance unit is configured to chart a path on the body surface of the subject from the current position of the ultrasound probe to the target location based on the current position of the ultrasound probe, the bounding boxes corresponding to the one or more detected anatomical regions of interest, the identified anatomical regions of interest, or combinations thereof.

17. The system of claim 16, wherein to communicate in real time the desired movement of the ultrasound probe the feedback unit is configured to provide a real-time indicator to a user, and wherein to provide the real-time indicator the feedback unit is configured to visualize the real-time indicator on a display, play an audio-indicator of the real-time indicator, or a combination thereof to guide the user to the target location.

18. An imaging system, the system comprising:
an acquisition subsystem configured to acquire at least one image corresponding to a subject;
a processing subsystem in operative association with the acquisition subsystem and configured to process the at least one image, wherein the processing subsystem comprises a navigation platform configured to:
determine in real-time a current position of an ultrasound probe on a body surface of the subject based on the at least one image;
identify in real-time one or more anatomical regions of interest in the at least one image;
quantify in real-time the at least one image to determine suitability of the at least one image to one or more scan planes corresponding to a determined clinical protocol;
generate in real-time a personalized anatomical model of the subject based on a current position of the ultrasound probe, previous positions of the ultrasound probe, the identified one or more anatomical regions of interest, and the quantification of the at least one image, or combinations thereof;
compute in real-time a desired trajectory of the ultrasound probe from the current location to a target location based on the determined clinical protocol; and
communicate in real-time a desired movement of the ultrasound probe based on the computed trajectory.

19. The imaging system of claim 18, wherein the acquisition subsystem is further configured to acquire images of the subject corresponding to movement of the ultrasound probe along the computed trajectory, and wherein the acquired images comprise a desired anatomical region of interest.

20. The imaging system of claim 18, further comprising a display configured to visualize the at least one image, the current location of the ultrasound probe, the quantification of the at least one image, the desired trajectory of the ultrasound probe, or combinations thereof.
